(19) United States
(12) Patent Application Publication
(10) Pub. No.: US 2007/0141695 A1
(43) Pub. Date: Jun. 21, 2007

(54) Luminescent dissolved oxygen sensor with visual verification

(75) Inventor: Thomas Owen Mitchell, Fort Collins, CO (US)

Correspondence Address: THE OLLILA LAW GROUP LLC, 2060 BROADWAY, SUITE 300, BOULDER, CO, 80302, US

(73) Assignee: HACH COMPANY

(21) Appl. No.: 11/312,197

(22) Filed: Dec. 20, 2005

(51) Int. Cl. C12M 3/00

(52) U.S. Cl. 435/287.2

(57) ABSTRACT

A method and apparatus for visually detecting when a luminescent dissolved oxygen sensor is operating is disclosed. In one example embodiment of the invention, a shutter is placed into the light tight container. When the shutter is open, a user can see into the light tight container and verify probe operation. When the shutter is closed, external light is prevented from entering the light tight container and affecting measurement accuracy. In another example embodiment of the invention, one end of a light pipe is placed on the outside of the light tight container, and the other end is positioned to view the light source of the probe. In another example embodiment of the invention a second light source, visible on the outside of the light tight container, is used to verify operation of the probe. In another example embodiment of the invention, a predetermined area is left open in the optically opaque hydrostatically transparent on the face of the sensor window, allowing a user to see light from the sensor when the sensor is operating properly.

### BACKGROUND OF THE INVENTION

1. FIELD OF THE INVENTION

The invention is related to the field of sensors, and in particular, to a luminescent dissolved oxygen sensor with a system and method for visual verification.

2. STATEMENT OF THE PROBLEM

The concentration of oxygen in water can be measured with a probe. The oxygen in the water interacts with a luminescent material on the outside of the probe. This interaction between the oxygen and the luminescent material results in a phenomenon known as luminescent quenching. Thus, the amount of luminescent quenching indicates the concentration of oxygen in the water.

In operation, the probe directs a light source centered at one wavelength onto the luminescent material. The light causes the luminescent material to generate luminescent light centered at a different wavelength. Luminescence quenching affects the amount of time that the luminescent material continues to luminescence light. Thus, if the light source's signal varies sinusoidally, the luminescence quenching affects the phase shift between the excitation light and the luminescent light. The probe uses an optical sensor to measures the phase shift between the excitation light and the luminescent light to assess the amount of luminescent quenching. As a result, the probe processes the phase shift to determine the concentration of oxygen in the water. An example of such a probe is disclosed in U.S. Pat. No. 6,912,050 entitled “Phase shift measurement for luminescent light” filed Feb. 3, 2003, which is hereby incorporated by reference.

Sinusoidally varying the signal to the light source causes the light source to pulse on and off. In some probes, the light source is visible, allowing a user to determine when the probe is operating by viewing the pulsing light. Unfortunately, daylight hitting the luminescent material or the optical sensor can cause inaccuracies in the measurement of the concentration of oxygen in the water. Therefore the luminescent material and the optical sensor are now typically shielded from daylight. This may be done by enclosing the light source, the optical sensor, and the luminescent material inside a light tight container. The light tight container shields the light source in the probe from view and prevents a user from visually detecting when the probe is operating. Without a visual means for verifying that the probe is operating, the senor must be connected to a computer or other device to verify operation. A user may not have access to a computer when checking or installing the probe in the field. Even when the user has access to a computer, connecting the probe to a computer to verify probe operation takes more time than a simple visual verification.

Therefore there is a need for a system and method for allowing a user to visually detect when a luminescent dissolved oxygen sensor is operating.

### SUMMARY OF THE INVENTION

A method and apparatus for visually detecting when a luminescent dissolved oxygen sensor is operating is disclosed. In one example embodiment of the invention, a shutter is placed into the light tight container. When the shutter is open, a user can see into the light tight container and verify probe operation. When the shutter is closed, external light is prevented from entering the light tight container and affecting measurement accuracy. In another example embodiment of the invention, one end of a light pipe is placed on the outside of the light tight container, and the other end is positioned to view the light source of the probe. In another example embodiment of the invention a second light source, visible on the outside of the light tight container, is used to verify operation of the probe. In another example embodiment of the invention, a predetermined area is left open in the optically opaque hydrostatically transparent on the face of the sensor window, allowing a user to see light from the sensor when the sensor is operating properly.

### BRIEF DESCRIPTION OF THE DRAWINGS

FIG. 1 is a prior art example probe design with the luminescent material placed on the top of the sensor.

FIG. 2 is a cross-sectional side view of a probe 200 in an example embodiment of the invention.

FIG. 3 is a cross-sectional top view of a side view probe 300 in an example embodiment of the invention.

FIG. 4 is an isometric view of a sensor board 400 that uses a light pipe for visual verification of probe operation in an example embodiment of the current invention.

FIG. 5 is a cross-sectional view of a probe with a light pipe in an example embodiment of the invention.

FIG. 6 is a detailed view of a probe with a second light source in an example embodiment of the invention.

FIG. 7 is an isometric view of a sensor cap in an example embodiment of the invention.

FIG. 8 is a cross-sectional view of a sensor window in an example embodiment of the invention.

### DETAILED DESCRIPTION OF THE PREFERRED EMBODIMENT

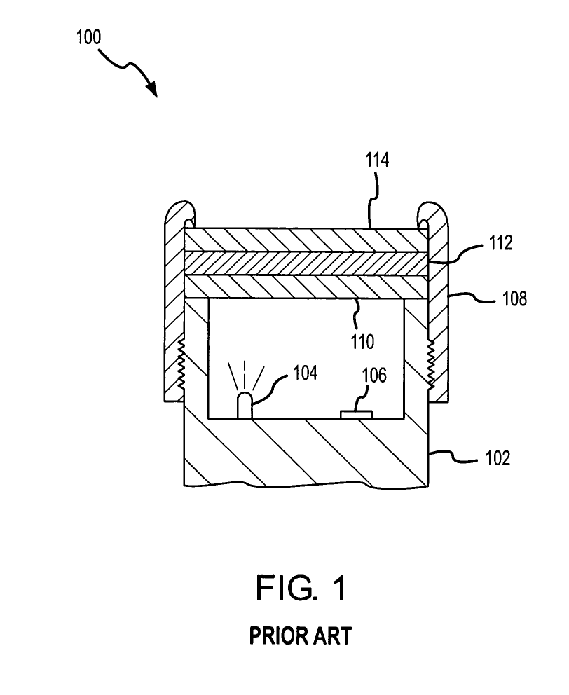

FIGS. 1-8 and the following description depict specific examples to teach those skilled in the art how to make and use the best mode of the invention. For the purpose of teaching inventive principles, some conventional aspects have been simplified or omitted. Those skilled in the art will appreciate variations from these examples that fall within the scope of the invention. Those skilled in the art will appreciate that the features described below can be combined in various ways to form multiple variations of the invention. As a result, the invention is not limited to the specific examples described below, but only by the claims and their equivalents.

Luminescent dissolved oxygen sensors (also called probes) can be made using a number of different layouts. Some sensors place the luminescent material on the end of the sensor and some place the luminescent material on the side of the sensor. Sensors with different layouts typically have a number of common design elements. FIG. 1 is an example probe design with the luminescent material placed on the top of the sensor. Probe 100 comprises probe body 102, light source 104, optical detector/sensor 106, retaining cap 108, hydrostatic barrier 110, luminescent material 112, and optically opaque hydrostatically transparent material 114. Luminescent material 112 is typically a mix of Polystyrene and Platinum Porphynin. Optically opaque hydrostatically transparent materials allow fluids to penetrate the material but block light from penetrating the material. One example of an optically opaque hydrostatically transparent material is a mix of carbon lamp black and Polybutyl Methacrylate. The drawings are not to scale and some thicknesses have been increased for clarity in explaining the invention, for example, in practice the optically opaque hydrostatically transparent material may only be a thin layer (10-20 microns) deposited over the other layers.

Body 102 contains light source 104 and optical detector/sensor 106 as well as electronics (not shown) used to drive the light source 104 and the optical detector 106. Light source 104, optical sensor 106, and electronics typically need to be kept dry. A hydrostatic barrier 110 forms a seal against body 102 to prevent fluids from entering the cavity formed by body 102. An O-ring or gasket (not shown) may be used to help form the seal between the hydrostatic barrier 110 and the body 102. The hydrostatic barrier 110 can be made from any material that is optically transparent and hydrostatically opaque, for example plastic, glass, crystal, or the like. The luminescent material 112 is placed on top of the hydrostatic barrier 110. An optically opaque hydrostatically transparent material 114 is placed on top of the luminescent material 112. A retaining cap 108 is used to hold the hydrostatic barrier 110, luminescent material 112, and optically opaque hydrostatically transparent material 114 onto the body 102. The retaining cap is made from an optically opaque material or coated with an optically opaque material. The body 102, the retaining cap 108, and the optically opaque hydrostatically transparent material 114 form a light tight container around light source 104, optical detector 106, and luminescent material 112.

In operation, the probe is immersed in water. The optically opaque hydrostatically transparent material 114 allows water to penetrate to the luminescent material 112. Hydrostatic barrier 110 prevents the water from entering the cavity formed by the body 102. The wet luminescent material is illuminated by light source 104 through hydrostatic barrier 110. The luminescent material 112 emits light in response to the illumination from light source 104. The duration of the response is dependent on the concentration of oxygen in the water. Optical sensor 106 detects the light emitted from the luminescent material 112.

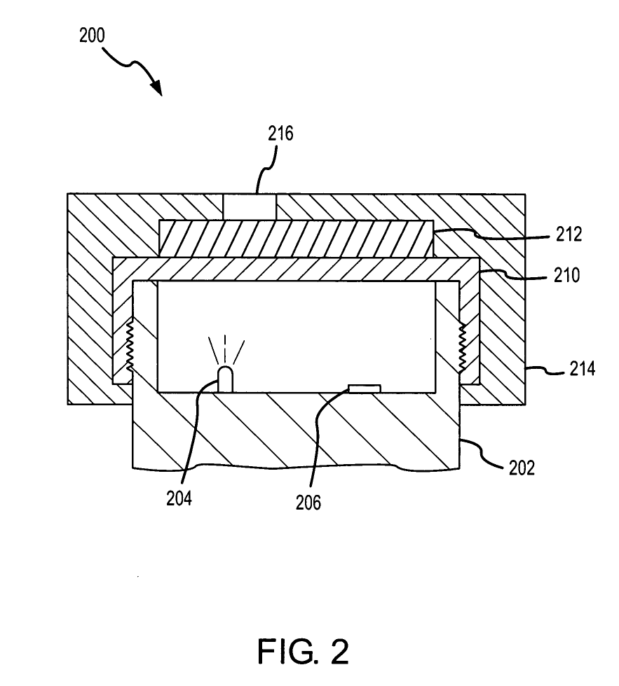

FIG. 2 is a cross-sectional side view of a probe 200 in an example embodiment of the invention. Probe 200 comprises probe body 202, light source 204, optical detector/sensor 206, hydrostatic barrier 210, luminescent material 212, optically opaque hydrostatically transparent material 214, and shutter 216.

Body 202 contains light source 204 and optical detector/sensor 206 as well as electronics (not shown) used to drive the light source and the optical detector 206. Light source 204, optical sensor 106, and electronics typically need to be kept dry. A hydrostatic barrier 210 forms a seal against body 202 to prevent fluids from entering the cavity formed by body 202. An O-ring or gasket (not shown) may be used to help form the seal between the hydrostatic barrier 210 and the body 202. The hydrostatic barrier 210 can be made from any material that is optically transparent and hydrostatically opaque, for example plastic, glass, crystal, or the like. The hydrostatic barrier is shaped as a cap that screws onto body 202. The luminescent material 212 is placed on top of the hydrostatic barrier 210. An optically opaque hydrostatically transparent 214 material is placed on top of the luminescent material 212 and surrounds hydrostatic barrier 210. The body 202 and the optically opaque hydrostatically transparent material 214 form a light tight container around light source 204, optical detector 206, and luminescent material 212. Shutter 216 is placed on top of luminescent material 212. Shutter can be opened or closed. When closed, shutter is optically opaque. When open, shutter is optically transparent and allows light from luminescent material 212 or from light source 204 to exit the light tight container and be seen by a user, allowing visual verification of probe 200 operation. In one example embodiment of the invention, a user would open the shutter and visually verify that the probe was operating. Once the user has visually verified that the probe was operating, the user would close the shutter. Shutter 216 can be an optical shutter, for example a liquid crystal shutter, a mechanical shutter, or the like.

Mechanical shutters are well known in the arts. Any type of mechanical shutter can be used as shutter 216. Some of the possible embodiments include a sliding or rotating door, a rotating vain, a folded flexible material (like Venetian blinds), an iris, or the like. Shutter 216 can be manually operated or power driven, for example using an electro-magnetic force. Shutter 216 is not required to be located on top of luminescent material 212. Shutter 216 can be located anywhere in the perimeter of the light tight container such that when the shutter is open it allows light to exit the light tight container.

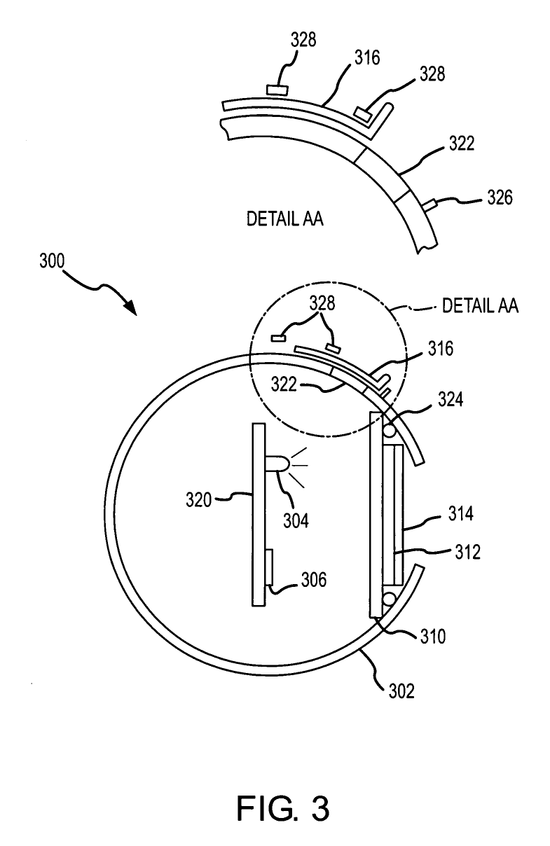

FIG. 3 is a cross-sectional top view of a side view probe 300 in an example embodiment of the invention. Side view probes sense a condition through the side of the probe, not through the top of the probe. Side view probe 300 comprises probe body 302, printed circuit (PC) board 320, light source 304, optical sensor 306, hydrostatic barrier 310, luminescent material 312, optically opaque hydrostatically transparent material 314, O-ring seal 324, mechanical shutter 316, and window 322. Probe body 302 is shown as a circle, but may take any shape. PC board 320 is mounted inside probe body 302. Light source 304 and optical sensor 306 are mounted on PC board 320 and face an opening in probe body 302. Hydrostatic barrier 310 is mounted in the opening in probe body 302. O-ring 324 helps form a seal between probe body 302 and hydrostatic barrier 310. Luminescent material 312 is attached to the outside of hydrostatic barrier 310. Optically opaque hydrostatically transparent material 314 is attached to the outside of luminescent material 312 and forms a light tight container with probe body 302. Window 322 is installed into probe body 302. Mechanical shutter 316, when closed (as shown in FIG. 3) covers window 322 and prevents light from being transmitted through window 322. Mechanical shutter 316, when open (as shown in detail AA) does not cover window 322 and allows light to be transmitted through window 322. Mechanical shutter 316 is retained by clips 328 and may include stop 326. Mechanical shutter 316 may have a latch or feature (not shown) that holds the shutter in the open or closed position. Mechanical shutter 316 or the mechanical shutter 316 and window 322 combination may be replaced by an optical shutter. Mechanical shutter 316 is shown as a sliding door type mechanical shutter. But as discussed above, any type of mechanical shutter may be used. In operation, shutter 316 is opened to allow a visual verification that probe 300 is operating.

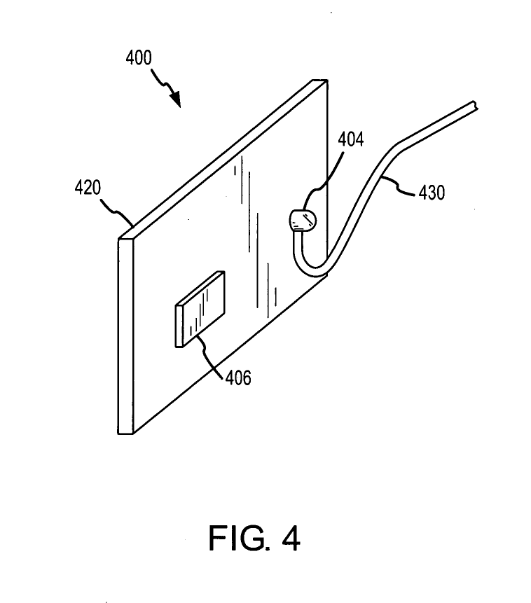

FIG. 4 is an isometric view of a sensor board 400 that uses a light pipe for visual verification of probe operation in an example embodiment of the current invention. Sensor board comprises PC board 420, optical sensor 406, light source 406, and light pipe 420. Optical sensor 406 and light source 404 are mounted onto PC board 420. Light pipe 420 is typically made from an optical fiber. The optical fiber may be clad or un-clad. The ends of the optical fiber may have a lens attached to increase or decrease the exit pupil of the fiber.

In operation the PC board is mounted inside a probe with the light source and the optical sensor facing a luminescent material. A first end of light pipe 430 is directed towards light source 404. The first end of the light pipe 430 may be clamped, held or glued in place. The optical axis of the first end light pipe 430 may be directed towards the light source with an orientation directed away from the optical sensor 406 and directed towards the PC board. With this orientation, any light that exits the first end of the light pipe is directed away from the optical sensor and away from the luminescent material. In one example embodiment of the invention, the optical axis of the light pipe is perpendicular to a line running between the light source and the optical sensor. When sensor board 400 is installed into a probe, the second end of the light pipe is mounted such that it can be seen on the outside of the light tight container. This allows a user to look at the second end of the light pipe and determine when the light source in the probe is functioning. A band pass filter (not shown) corresponding to the wavelength of the light source may optionally be attached to either end of the light pipe. The band pass filter would prevent any light not corresponding to the wavelength of the light source from being transmitted through the light pipe. This would limit the amount of external light entering the probe through the light pipe.

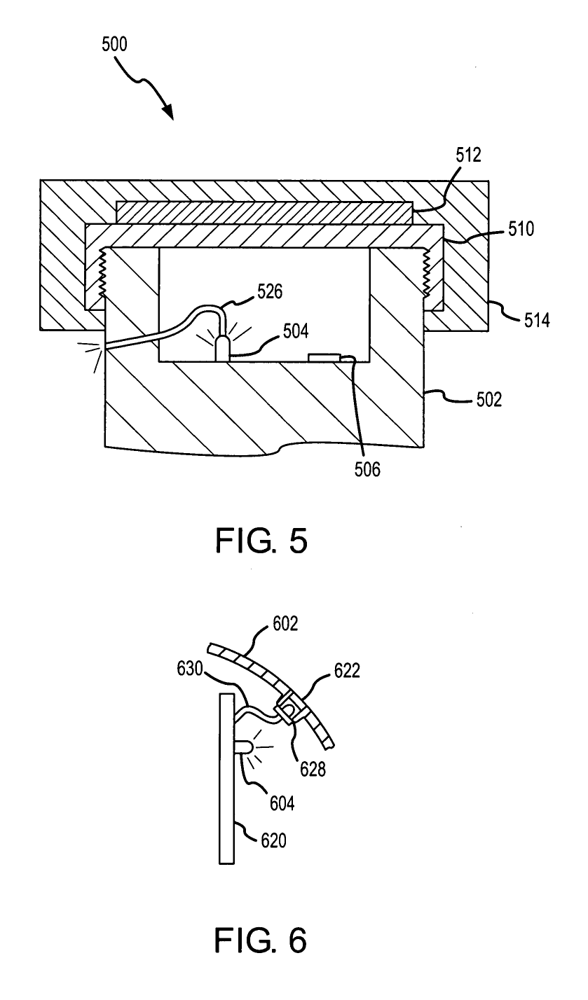

FIG. 5 is a cross-sectional view of a probe using a light pipe in an example embodiment of the invention. Probe 500 comprises probe body 502, light source 504, optical detector/sensor 506, hydrostatic barrier 510, luminescent material 512, optically opaque hydrostatically transparent material 514, and light pipe 226.

Body 502 contains light source 504 and optical detector/sensor 506 as well as electronics (not shown) used to drive the light source 504 and the optical detector 506. The hydrostatic barrier is shaped as a cap that screws onto body 502. The luminescent material 512 is placed on top of the hydrostatic barrier 510. An optically opaque hydrostatically transparent 514 material is placed on top of the luminescent material 512 and surrounds hydrostatic barrier 510. The body 502 and the optically opaque hydrostatically transparent material 514 form a light tight container around light source 504, optical detector 506, and luminescent material 512. A first end of light pipe 526 is directed towards light source 504. The second end of light pipe 526 is mounted such that it is visible from the outside of the light tight container.

In another example embodiment of the invention, the probe would have two light sources. The first light source would be used to illuminate the luminescent material, and the second light source would be use to signal the user that the probe was operating. The second light source would be mounted such that the light from the second source would be visible outside the light tight container while preventing external light from entering the light tight container. In one example embodiment of the invention, the second light source 628 would fit into an opening in the body of the probe (see FIG. 6). A window 622 may be used to help form a water tight seal above the second light source 628 or the second light source 628 may be sealed inside the opening forming a water tight seal. The second light source may be powered from the same PC board as the first light source or may be powered from another source. The second light source may pulse on and off when the probe is operating or may be set to a constant illumination when the probe is operating. The second light source 628 may be coupled to the PC board 620 with a flex cable, wire 630, or the like, or may be surface mounted onto the PC board 620. In another example embodiment of the invention, one end of a light pipe would be mounted directly on top of the second light source with the second end of the light pipe mounted such that it can be seen on the outside of the probe. With the light pipe mounted directly over the second light source, stray light entering the light pipe would be prevented from reaching the luminescent material or the optical sensor.

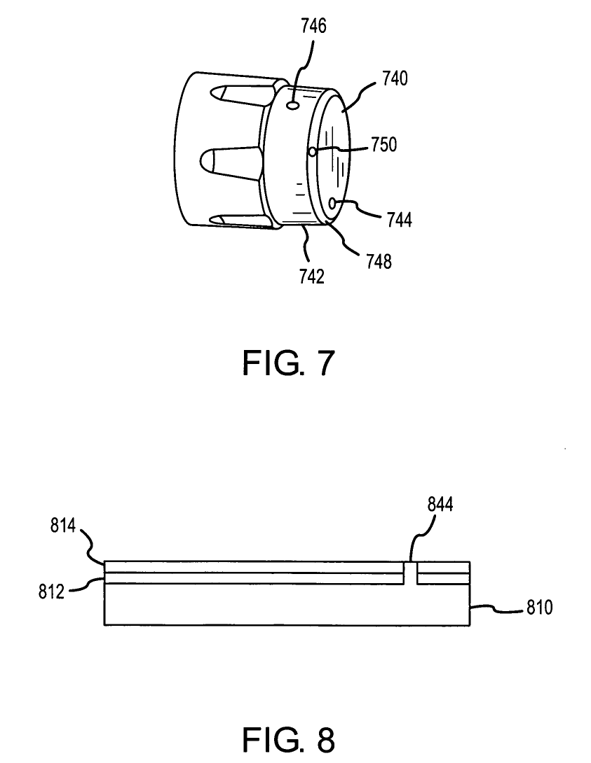

FIG. 7 is an isometric view of a sensor cap in an example embodiment of the invention. The sensor cap comprises a flat sensor face 740, chamfer 748, and side face 742. Underneath the flat sensor face 740 are three layers. The top layer is an optically opaque hydrostatically transparent material. The middle layer is a luminescent material. And the bottom layer is a hydrostatic barrier. The luminescent material typically only covers the flat sensor face 740. In the past the optically opaque hydrostatically transparent material only covered the luminescent material deposited on the flat face 740. The side face and the chamfer were left uncovered by the optically opaque hydrostatically transparent material. The uncovered side face 742 and chamfer 740 allowed too much light to penetrate into the sensor. The current practice is to cover the flat sensor face, the chamfer, and the side face 742 with the optically opaque hydrostatically transparent material. This prevents outside light from penetrating into the sensor, but also prevents light from the sensor to be seen by a user to verify sensor operation.

In one example embodiment of the invention, at least one small area is left uncovered by the optically opaque hydrostatically transparent material. The uncovered or exposed area can be on the flat sensor face, for example small area 744. The uncovered or exposed area can be on the chamfer, for example small area 750. The uncovered or exposed area can be on the side face, for example small area 746. In one embodiment of the invention, there are a plurality of uncovered or exposed areas distributed at different places on the sensor cap. By limiting the uncovered or exposed area to a small portion of the total area of the sensor, the effect on the accuracy of the sensor can be minimized while providing the user with visual verification that the sensor is operating properly. In one example embodiment of the invention, the exposed or uncovered area is smaller than 5% of the total sensor area, where the total sensor area is the area covered by the luminescent material. The position or location of the uncovered area may allow the size of the area to be increased. When the uncovered area is placed in a location as far away from the optical sensor as possible, the size of the uncovered area may be increased. The uncovered areas can be formed by removing the optically opaque hydrostatically transparent material or by masking small areas during the application of the optically opaque hydrostatically transparent material to the sensor cap.

FIG. 8 is a cross-sectional view of a sensor window in an example embodiment of the invention. Sensor window comprises an optically opaque hydrostatically transparent material 814, a luminescent material 812, a hydrostatic barrier 810, and a small exposed area 844. The small exposed area is formed by a column or protrusion of the hydrostatic barrier that sticks up through the luminescent material 812 and through the optically opaque hydrostatically transparent material 814. Using this method allows the small uncovered or exposed area to be flush with the top surface of the sensor window. One small area or multiple smaller areas may be formed in this manner.

We claim:

1. A luminescent dissolved oxygen sensor, comprising:
a light tight container having an inside and an outside;
an optically opaque hydrostatically transparent material forming at least one section of the light tight container;
a luminescent material on the inside of the light tight container having a first side and a second side, where the first side contacts the optically opaque hydrostatically transparent material;
a hydrostatic barrier contacting the second side of the luminescent material;
a light source located on the inside of the light tight container and configured to illuminate the luminescent material through the hydrostatic barrier;
a shutter in the light tight container, the shutter, when open, configured to allow light to exit the light tight container.

2. The luminescent dissolved oxygen sensor of claim 1 where the shutter is an optical shutter.

3. The luminescent dissolved oxygen sensor of claim 1 where the shutter is a mechanical shutter.

4. The luminescent dissolved oxygen sensor of claim 3 where the shutter further comprises:
a sliding panel movable between an open position and a closed position.

5. The luminescent dissolved oxygen sensor of claim 3 where the shutter further comprises:
an iris movable between an open position and a closed position.

6. The luminescent dissolved oxygen sensor of claim 3 where the shutter further comprises:
a rotating panel movable between an open position and a closed position.

7. The luminescent dissolved oxygen sensor of claim 3 further comprising:
a window mounted under the shutter where the window forms part of a water tight container with the light source inside the water tight container.

8. The luminescent dissolved oxygen sensor of claim 1 where the luminescent material is on an end of the luminescent dissolved oxygen sensor.

9. The luminescent dissolved oxygen sensor of claim 1 where the luminescent material is on a side of the luminescent dissolved oxygen sensor.

10. The luminescent dissolved oxygen sensor of claim 1 where shutter is manually operated.

11. A method, comprising:
opening a shutter on a luminescent dissolved oxygen sensor;
determining that the sensor is operating when light can be seen through the open shutter;
closing the shutter.

12. A luminescent dissolved oxygen sensor, comprising:
a light tight container having an inside and an outside;
an optically opaque hydrostatically transparent material forming at least one section of the light tight container;
a luminescent material on the inside of the light tight container having a first side and a second side, where the first side contacts the optically opaque hydrostatically transparent material;
a hydrostatic barrier contacting the second side of the luminescent material;
a light source located on the inside of the light tight container and configured to illuminate the luminescent material through the hydrostatic barrier;
a light pipe having a first end and a second end where the first end is directed towards the light source and the second end is visible on the outside of the light tight container.

13. The luminescent dissolved oxygen sensor of claim 12 where the luminescent material is on an end of the luminescent dissolved oxygen sensor.

14. The luminescent dissolved oxygen sensor of claim 12 where the luminescent material is on a side of the luminescent dissolved oxygen sensor.

15. A luminescent dissolved oxygen sensor, comprising:
a light tight container having an inside and an outside;
an optically opaque hydrostatically transparent material forming at least one section of the light tight container;
a luminescent material on the inside of the light tight container having a first side and a second side, where the first side contacts the optically opaque hydrostatically transparent material;
a hydrostatic barrier contacting the second side of the luminescent material;
a light source located on the inside of the light tight container and configured to illuminate the luminescent material through the hydrostatic barrier;
a second light source configured to be seen on the outside of the light tight container.

16. The luminescent dissolved oxygen sensor of claim 15 where the second light source is mounted in an opening in the light tight container.

17. The luminescent dissolved oxygen sensor of claim 15 where a first end of a light pipe is mounted directly over the second light source and a second end of the light pipe is visible outside the light tight container.

18. The luminescent dissolved oxygen sensor of claim 15 where the light from the second light source does not illuminate the inside of the light tight container.

19. A luminescent dissolved oxygen sensor, comprising:
a light tight container;
a luminescent material inside the light tight container;
a light source inside the light tight container and configured to illuminate the luminescent material;
means for switchably allowing light to exit the light tight container.

20. A luminescent dissolved oxygen sensor, comprising:
a sensor window comprising an outer layer, a middle layer and an inner layer;
the outer layer comprising an optically opaque hydrostatically transparent material;
the middle layer comprising a luminescent martial;
the inner layer comprising a hydrostatic barrier;
at least one small void formed in the outer layer that is configured to pass light through the outer layer.

21. The luminescent dissolved oxygen sensor of claim 1 where the at least one small void is smaller than 5% of a total area of the sensor window.

22. The luminescent dissolved oxygen sensor of claim 1 where a small column of the inner layer extends from the inner layer through the middle layer and through the outer layer, filling the at least one small void formed in the outer layer.

23. The luminescent dissolved oxygen sensor of claim 22 where the top surface of the sensor window is essentially flat.

24. A method, comprising:
coating a sensor window area on a hydrostatic barrier with a luminescent material;
coating the luminescent material with an optically opaque hydrostatically transparent material;
removing a small area of the optically opaque hydrostatically transparent material from the sensor window area.

25. A method, comprising:
coating a sensor window area on a hydrostatic barrier with a luminescent material;
coating the luminescent material with an optically opaque hydrostatically transparent material;
exposing a small area of the hydrostatic barrier through the optically opaque hydrostatically transparent material and the luminescent material.
